(19) United States
(12) Patent Application Publication
(10) Pub. No.: US 2014/0259320 A1
(43) Pub. Date: Sep. 18, 2014

(54) AIR SAFETY PROTECTOR FOR THE FACIAL AREA INCLUDING THE EYES, NOSE, MOUTH AND EARS

(71) Applicants: Oscar Gonzalez, San Antonio, TX (US); Francisco Iriarte, San Antonio, TX (US)

(72) Inventors: Oscar Gonzalez, San Antonio, TX (US); Francisco Iriarte, San Antonio, TX (US)

(21) Appl. No.: 14/213,125

(22) Filed: Mar. 14, 2014

Related U.S. Application Data
(60) Provisional application No. 61/787,498, filed on Mar. 15, 2013.
(60) Provisional application No. 61/886,522, filed on Oct. 3, 2013.

(51) Int. Cl. A61F 9/02

(52) U.S. Cl. CPC A61F 9/028
USPC 2/424

(57) ABSTRACT

A device and method of use for protection of the facial area including but not limited to the eyes, nose, mouth, and ears through the use of airflow to create a protective barrier for this area is disclosed. In one embodiment, the device for providing protection of the facial area through the use of airflow to create a protective barrier for this area is comprised of an air delivery member, a frame member, and an air nozzle member. In another embodiment, the device is also comprised of an air supply member. In another embodiment, the device is also comprised of a control member. In yet another embodiment, the air nozzle member is comprised of one or more air nozzles that may be of varying nozzle shapes to control the air barrier produced. The air nozzle member may also be positioned in various directions and the exit velocity of the air leaving the nozzle controlled via the control member. Lastly, in another embodiment the device may also be comprised of a lens member.

### CROSS-REFERENCE TO RELATED APPLICATIONS

This application claims the benefit under Title 35 United States Code § 119(e) of U.S. Provisional Patent Application Ser. No. 61/787,498; Filed: Mar. 15, 2013, the full disclosure of which is incorporated herein by reference and of U.S. Provisional Patent Application Ser. No. 61/886,522; Filed: Oct. 3, 2013, the full disclosure of which is also incorporated herein by reference.

### STATEMENT REGARDING FEDERALLY SPONSORED RESEARCH OR DEVELOPMENT

Not applicable

### THE NAMES OF THE PARTIES TO A JOINT RESEARCH AGREEMENT

Not applicable

### INCORPORATING-BY-REFERENCE OF MATERIAL SUBMITTED ON A COMPACT DISC

Not applicable

### SEQUENCE LISTING

Not applicable

### FIELD OF THE INVENTION

The present invention generally relates to a device and method of use directed to protection for the facial area. More specifically, the present invention relates to a device and method of use for protection of the facial area to include but not be limited to the eyes, nose, mouth, and ears through the use of airflow to create a protective barrier.

### BACKGROUND OF THE INVENTION

Without limiting the scope of the disclosed device and method, the background is described in connection with a novel device and approach directed to protecting the facial area through the use of airflow to create a protective barrier for this area.

In work environments such as construction, individuals may have their facial area such as their eyes, nose, mouth, and ears exposed to a number of different types of substances in solid, liquid, and gas forms. An individual applying paint to a surface via sprayers may have paint splash or mist into their eyes, nose, mouth, and/or ears. In addition, the same issues arise when individuals are applying foam insulation, airless spray coatings, chemicals, and powders.

Currently, the facial protection methods utilized are safety glasses, face masks, and variations of solid materials such as glass and polymers to form a rigid clear barrier in front of the individual's facial area. However, while these methods do offer some protection, they suffer from several drawbacks. Often, these existing protectors get stained, warped, and constantly need to get wiped and/or cleaned during use. The constant need to wipe and/or clean the existing state of the art devices lends itself to increased maintenance, lost productivity, and increased costs. In addition, working with the current state of the art's existing facial protectors while dirty, exposes the individual to possible accidents and less than optimal work quality output.

While all of the aforementioned approaches may fulfill their unique purposes, none of them fulfill the need for a practical and effective means for providing facial area protection including but not limited to the eyes, nose, mouth, and ears.

The present invention therefore proposes a novel device and method of use for providing facial area protection through the use of airflow to create a protective barrier for this area.

### BRIEF SUMMARY OF THE INVENTION

The present invention, therefore, provides a device and method of use directed to protecting the facial area through the use of airflow to create a protective barrier for this area.

In one embodiment, the device for providing facial area protection through the use of airflow to create a protective barrier for this area is comprised of an air delivery member, a frame member, and an air nozzle member. In another embodiment, the device is also comprised of an air supply member. In another embodiment, the device is also comprised of a control member. In yet another embodiment, the air nozzle member is comprised of one or more air nozzles that may be of varying nozzle shapes to control the air barrier produced. The air nozzle member may also be positioned in various directions and the exit velocity of the air leaving the nozzle controlled via the control member. Lastly, in another embodiment the device may also be comprised of a lens member.

In summary, the present invention discloses an improved device and method of use directed protection of the facial area. More specifically, the present invention relates to a device and method of use for protection of the facial area to include but not be limited to the eyes, nose, mouth, and ears through the use of airflow to create a protective barrier.

### BRIEF DESCRIPTION OF THE SEVERAL VIEWS OF THE DRAWINGS

For a more complete understanding of the features and advantages of the present invention, reference is now made to the detailed description of the invention along with the accompanying figures in which:

FIG. 1 is a front perspective view of the air safety protector for the face area device in accordance with embodiments of the disclosure;

FIG. 2 is a side view of the air safety protector for the face area device in accordance with embodiments of the disclosure;

FIG. 3 is a top view of the air safety protector for the face area device in accordance with embodiments of the disclosure;

FIG. 4 is a rear perspective view of the air safety protector for the face area device in accordance with embodiments of the disclosure;

FIG. 5 is a front view of the air safety protector for the face area device positioned on a user in accordance with embodiments of the disclosure;

FIG. 6 is a side perspective view of the air safety protector for the face area device positioned on a user in accordance with embodiments of the disclosure;

FIG. 7 is a front perspective view of the air safety protector for the face area device illustrating an alternative embodiment for the air nozzle member in accordance with embodiments of the disclosure;

FIG. 8 is a front perspective view of the air safety protector for the face area device illustrating an alternative embodiment without the lens member in accordance with embodiments of the disclosure.

### DETAILED DESCRIPTION OF THE INVENTION

Disclosed herein is an improved device and method of use for protection of the face area through the use of airflow to create a protective barrier for this area. The numerous innovative teachings of the present invention will be described with particular reference to several embodiments (by way of example, and not of limitation).

Reference is first made to FIG. 1, a front perspective view of the air safety protector for the facial area device in accordance with embodiments of the disclosure. The facial area is considered to include but not be limited to the eyes, nose, ears, and mouth area. That is the front and side areas of an individual's head. In one embodiment, the device for providing facial area protection through the use of airflow to create a protective barrier for this area is comprised of an air delivery member 10, a frame member 20, and an air nozzle member 30. The air delivery member 10 provides a channel through which air or a gas may flow through to reach the air nozzle member 30. The air delivery member 10 has a connection point 60 that is used to connect the air delivery member 10 to an air supply member. This connection or attachment may be by several means such as but not limited to threaded connections, magnetic connections, and locking means. The air supply member may be an air compressor or other source of air such as a duct fan or other mean of generating air flow through the air delivery member 10. The air supply member may be attached to the user via a backpack, belt, or moved about by portable means such as rollers. The frame member 20 is the structure that provides attachment means to the user and may connect to the air delivery member 10 and/or the air nozzle member 30. The frame member 20 may be similar to an eye glass frame as depicted, a headband, a hat, a helmet, or other similar structure for positioning the air nozzle member 30 around the facial area. The air nozzle member 30 is the location where the supplied air exits 70 the device to create an air barrier in the facial area of the user. Air nozzle members 30 may be placed on the side of the frame member 20 or the air delivery member 10 to provide protection for the ear area or side of the individual's head. The air nozzle member 30 may be comprised of one nozzle or many nozzles and the nozzles may have various nozzle opening shapes. The nozzles that make up the nozzle member 30 may be positioned in various configurations/layouts. The air nozzle member 30 may have the ability to control the direction of air flow 70 and the magnitude/velocity of the air flow 70. These flexible configurations (number of, shapes, and configuration layouts) of the nozzles, air flow 70 direction, and air flow velocity allow the device to provide various custom airflow patterns to be generated to protect the user's eyes for various environment conditions. For example, multiple nozzles across in a horizontal line may be directed towards the center or nose of the user creating a diamond pattern. The device for providing protection of the facial area through the use of airflow to create a protective barrier for this area may also be comprised of a control member 40. The control member 40 may have controls to set the direction of air flow for the air nozzle member 30. In addition, the control member 40 may have controls to control the air pressure being supplied by the air supply member and/or velocity of the air flow 70 leaving the air nozzle member 30. These settings are important for adjusting the device for proper environmental conditions. For instance, the air barrier protection level can be adjusted by positioning the air nozzles horizontal to the individual for contaminates falling from the ceiling. Or the air nozzles can be positioned vertically down to the individual for contaminates coming at the individual in a horizontal direction. Adjusting the air nozzle air flow 70 velocity for the size and density of the contaminate is important as well. Lighter contaminates such as dust will require a lower air nozzle air flow 70 velocity setting than a heavier contaminate such as paint. The device for providing protection of the facial area through the use of airflow to create a protective barrier for this area may also be comprised of a lens member 50. The lens member 50 provides a means of rigid protection and may be in the structure or similar structure of goggle lenses, eye glass lenses, or a face shield.

Reference is now made to FIG. 2, a side view of the air safety protector for the facial area device in accordance with embodiments of the disclosure. In this illustration, the air nozzle member 30 is shown being adjusted in the up and down direction steering the exit air 70 direction.

Reference is next made to FIG. 3, a top view of the air safety protector for the facial area device in accordance with embodiments of the disclosure. In this illustration, the connection points 60 are shown on each air delivery member 10. That is, there are different configurations for the connection points and there may be one or more connection points at which air is being supplied from an air supply member.

Reference is next made to FIG. 4, a rear perspective view of the air safety protector for the facial area device in accordance with embodiments of the disclosure. In this illustration, the exit air flow 70 is shown in the horizontal direction.

Reference is next made to FIG. 5, a front view of the air safety protector for the facial area device positioned on a user in accordance with embodiments of the disclosure. In this illustration, the exit air flow 70 is shown in the down direction and the device is depicted on an individual. The air nozzle member 30 is depicted as a slit in the horizontal direction providing a more uniform laminar flow barrier of air.

Reference is next made to FIG. 6, a side perspective view of the air safety protector for the facial area device positioned on a user in accordance with embodiments of the disclosure. In this illustration, the device is depicted on an individual and shows how the frame member 20 and the air delivery member 10 may be structured to attach to an individual.

Reference is next made to FIG. 7, a front perspective view of the air safety protector for the facial area device illustrating an alternative embodiment for the air nozzle member 30 in accordance with embodiments of the disclosure. In this illustration, the air nozzle member 30 is comprised of multiple air nozzles that may have directional control for the exit air flow 70. The exit air flow 70 is depicted at a horizontal directional air flow.

Reference is lastly made to FIG. 8, a front perspective view of the air safety protector for the facial area device illustrating an alternative embodiment without the lens member 50 in accordance with embodiments of the disclosure. In this illustration, the device is shown in a base setup without the lens member 50.

In brief, as described herein provides for an effective and efficient protection of the facial area through the use of airflow to create a protective barrier for this area.

The disclosed device and method of use is generally described, with examples incorporated as particular embodiments of the invention and to demonstrate the practice and advantages thereof. It is understood that the examples are given by way of illustration and are not intended to limit the specification or the claims in any manner.

To facilitate the understanding of this invention, a number of terms may be defined below. Terms defined herein have meanings as commonly understood by a person of ordinary skill in the areas relevant to the present invention.

Terms such as “a”, “an”, and “the” are not intended to refer to only a singular entity, but include the general class of which a specific example may be used for illustration. The terminology herein is used to describe specific embodiments of the invention, but their usage does not delimit the disclosed device or method, except as may be outlined in the claims.

Alternative applications for this invention include using the device and method of use for protection of the facial area to include but not be limited to the eyes, nose, mouth, and ears in various environments and conditions. Consequently, any embodiments comprising a one piece or multi piece device having the structures as herein disclosed with similar function shall fall into the coverage of claims of the present invention and shall lack the novelty and inventive step criteria.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific device and method of use described herein. Such equivalents are considered to be within the scope of this invention and are covered by the claims.

All publications and patent applications mentioned in the specification are indicative of the level of those skilled in the art to which this invention pertains. All publications and patent application are herein incorporated by reference to the same extent as if each individual publication or patent application was specifically and individually indicated to be incorporated by reference.

In the claims, all transitional phrases such as “comprising,” “including,” “carrying,” “having,” “containing,” “involving,” and the like are to be understood to be open-ended, i.e., to mean including but not limited to. Only the transitional phrases “consisting of” and “consisting essentially of,” respectively, shall be closed or semi-closed transitional phrases.

The device and/or methods disclosed and claimed herein can be made and executed without undue experimentation in light of the present disclosure. While the device and methods of this invention have been described in terms of preferred embodiments, it will be apparent to those skilled in the art that variations may be applied to the device and/or methods and in the steps or in the sequence of steps of the method described herein without departing from the concept, spirit, and scope of the invention.

More specifically, it will be apparent that certain components, which are both shape and material related, may be substituted for the components described herein while the same or similar results would be achieved. All such similar substitutes and modifications apparent to those skilled in the art are deemed to be within the spirit, scope, and concept of the invention as defined by the appended claims.

What is claimed is:

1. A device for protecting the face area including the eyes, nose, mouth, and ears comprising:
an air delivery member configured to connect to an air supply member and deliver air supplied by said air supply member to at least one nozzle member;
a frame member configured to attach to the user of the device and is connected to said air delivery member and/or said nozzle member;
at least one said nozzle member configured to connect to said air delivery member for receiving said air supplied by the air supply member through said air delivery member.

2. The device of claim 1, wherein said device further comprises a control member.

3. The device of claim 1, wherein said device further comprises a lens member.

4. The device of claim 1, wherein said device further comprises an air supply member.

5. The device of claim 1, wherein said device further comprises a control member and a lens member.

6. . The device of claim 1, wherein said device further comprises a control member and an air supply member.

7. The device of claim 1, wherein said device further comprises a lens member and an air supply member.

8. The device of claim 1, wherein said device further comprises a control member, a lens, member, and an air supply member.

9. The device of claim 1, wherein said nozzle member has at least one said nozzle member providing a fixed directional air flow.

10. The device of claim 1, wherein said nozzle member has at least one said nozzle member providing adjustable directional air flow.

11. The device of claim 1, wherein said nozzle member is conical in shape.

12. The device of claim 1, wherein said nozzle member is a slit opening surrounding the outer facial area.

13. The device of claim 1, wherein said nozzle members are also positioned along the side of the user's head for protecting the ear area.

14. The device of claim 1, wherein said control member has controls to control the air flow direction of said air nozzle member.

15. The device of claim 1, wherein said control member has controls to control the air pressure being supplied by said supply member.

16. The device of claim 1, wherein said control member has controls to control the velocity of the air flow being supplied by said air nozzle member.

17. The device of claim 1, wherein said control member has controls to control the velocity of the air flow being supplied by said air nozzle member and controls to control the air pressure being supplied by said supply member.

18. The device of claim 1, wherein said device further comprises a control member, a lens, member, and an air supply member; wherein said nozzle member has at least one said nozzle member providing a fixed directional air flow; and wherein said control member has controls to control the velocity of the air flow being supplied by said air nozzle member and controls to control the air pressure being supplied by said supply member.

19. The device of claim 1, wherein said device further comprises a control member, a lens, member, and an air supply member; wherein said nozzle member has at least one said nozzle member providing adjustable directional air flow; and wherein said control member has controls to control the velocity of the air flow being supplied by said air nozzle member and controls to control the air pressure being supplied by said supply member.

20. The device of claim 1, wherein said device further comprises a control member, a lens, member, and an air supply member; wherein said nozzle member has at least one said nozzle member providing adjustable directional air flow; and wherein at least one said nozzle member is a slit opening surrounding the outer facial area; and wherein some said nozzle members are also positioned along the side of the user's head for protecting the ear area; and wherein said control member has controls to control the velocity of the air flow being supplied by said air nozzle member and controls to control the air pressure being supplied by said supply member.
